(19) United States
(12) Patent Application Publication
(10) Pub. No.: US 2021/0128776 A1
(43) Pub. Date: May 6, 2021

(54) NEGATIVE ION GENERATOR, WEARABLE AIR PURIFIER HAVING THE AFOREMENTIONED NEGATIVE ION GENERATOR, AND METHOD OF MANUFACTURING THE AFOREMENTIONED NEGATIVE ION GENERATOR

(71) Applicant: ible Technology Inc., Taipei City (TW)

(72) Inventors: Hung-Hsuan Chien, Taipei City (TW); Yu-Fan Tsai, New Taipei City (TW)

(21) Appl. No.: 16/805,807

(22) Filed: Mar. 1, 2020

(30) Foreign Application Priority Data
Oct. 31, 2019 (TW) 108214349

(51) Int. Cl. A61L 9/22; A62B 18/00; H01T 23/00; A62B 7/10

(52) U.S. Cl. CPC A61L 9/22; A62B 7/10; H01T 23/00; A62B 18/003

(57) ABSTRACT

A negative ion generator includes a fiber bundle, a boost circuit board, a sleeve component and an electrically conductive adhesive. The fiber bundle includes a combining portion. The boost circuit board is connected to the fiber bundle and includes an electrically conductive terminal. An output end of the electrically conductive terminal is inserted into the combining portion of the fiber bundle. The boost circuit board provides a high-voltage current to the fiber bundle to enable the fiber bundle to emit negative ions by corona discharging. An accommodating space is enclosed by the sleeve component. The combining portion of the fiber bundle is installed inside the accommodating space. The electrically conductive adhesive is poured into the accommodating space and located between the combining portion of the fiber bundle and the output end of the electrically conductive terminal for adhering and electrically connecting the fiber bundle to the electrically conductive terminal.

### BACKGROUND OF THE INVENTION

### 1. Field of the Invention

The present invention relates to an electronic device, and more particularly, to a negative ion generator, a wearable air purifier having the aforementioned negative ion generator, and a method of manufacturing the aforementioned negative ion generator.

### 2. Description of the Prior Art

With development of the society and advancement of the technology, a negative ion generator, which can provide negative ions, becomes more and more popular, and there are various related electronic devices, e.g., a negative ion air purifier or a negative ion hair dryer, available in the market. The negative ion generator usually includes a boost circuit board and a fiber bundle. The boost circuit board provides a high-voltage current to the fiber bundle to enable the fiber bundle to emit negative ions by corona discharging. Please refer to FIG. 1 to FIG. 3. FIG. 1 and FIG. 2 are partial diagrams of a negative ion generator 2 at different views in the prior art. FIG. 3 is a partial exploded diagram of the negative ion generator 2 in the prior art. As shown in FIG. 1 to FIG. 3, a fiber bundle 21 and an output terminal 22 of a boost circuit board 20 of the negative ion generator 2 are clipped together by a metal clip 23, so that the fiber bundle 21 is electrically connected to the boost circuit board 20 by a clipping engagement of the output terminal 22 and the fiber bundle 21. However, sometimes, such fixing mechanism causes failure of emission of negative ions due to a poor contact between the boost circuit board 20 and the fiber bundle 21, i.e., such fixing mechanism has a poor electrically conductive efficiency between the boost circuit board 20 and the fiber bundle 21. Therefore, it becomes an important topic to improve both of a structural strength of a connection of the boost circuit board and the fiber bundle and an electrically conductive efficiency between the boost circuit board and the fiber bundle of the conventional negative ion generator.

### SUMMARY OF THE INVENTION

It is an objective of the present invention to provide a negative ion generator, a wearable air purifier having the aforementioned negative ion generator, and a method of manufacturing the aforementioned negative ion generator, which have an improved structural strength of a connection of a boost circuit board and a fiber bundle and an improved electrically conductive efficiency between the boost circuit board and the fiber bundle, for solving the aforementioned problems.

In order to achieve the aforementioned objective, the present invention discloses a negative ion generator. The negative ion generator includes a fiber bundle, a boost circuit board, a sleeve component and an electrically conductive adhesive. The fiber bundle includes a combining portion. The boost circuit board is connected to the fiber bundle. The boost circuit board includes an electrically conductive terminal. An output end of the electrically conductive terminal is inserted into the combining portion of the fiber bundle. The boost circuit board is for providing a high-voltage current to the fiber bundle to enable the fiber bundle to emit negative ions by corona discharging. An accommodating space is enclosed by the sleeve component. The combining portion of the fiber bundle is installed inside the accommodating space. The electrically conductive adhesive is poured into the accommodating space and located between the combining portion of the fiber bundle and the output end of the electrically conductive terminal for adhering and electrically connecting the fiber bundle to the electrically conductive terminal by the electrically conductive adhesive.

According to an embodiment of the present invention, the sleeve component is a hollow pipe structure.

According to an embodiment of the present invention, the sleeve component is made of plastic material.

According to an embodiment of the present invention, the fiber bundle includes a plurality of fibers.

According to an embodiment of the present invention, each of the plurality of fibers is made of carbon material.

According to an embodiment of the present invention, the electrically conductive adhesive includes silver powders and thermosetting plastic.

In order to achieve the aforementioned objective, the present invention further discloses a wearable air purifier. The wearable air purifier includes a wearable component and a negative ion generator detachably connected to the wearable component. The negative ion generator includes a fiber bundle, a boost circuit board, a sleeve component and an electrically conductive adhesive. The fiber bundle includes a combining portion. The boost circuit board is connected to the fiber bundle. The boost circuit board includes an electrically conductive terminal. An output end of the electrically conductive terminal is inserted into the combining portion of the fiber bundle. The boost circuit board is for providing a high-voltage current to the fiber bundle to enable the fiber bundle to emit negative ions by corona discharging. An accommodating space is enclosed by the sleeve component. The combining portion of the fiber bundle is installed inside the accommodating space. The electrically conductive adhesive is poured into the accommodating space and located between the combining portion of the fiber bundle and the output end of the electrically conductive terminal for adhering and electrically connecting the fiber bundle to the electrically conductive terminal by the electrically conductive adhesive.

According to an embodiment of the present invention, the wearable component is a necklace, a collar or a wristband.

In order to achieve the aforementioned objective, the present invention further discloses a method of manufacturing a negative ion generator. The method includes sheathing a sleeve component onto a fiber bundle, so as to locate a combining portion of the fiber bundle at an accommodating space enclosed by the sleeve component; inserting an output end of an electrically conductive terminal of a boost circuit board into the combining portion of the fiber bundle; and pouring an electrically conductive adhesive into the accommodating space, so as to adhere the fiber bundle to the electrically conductive terminal for electrically connecting the electrically conductive terminal to the fiber bundle by the electrically conductive adhesive.

According to an embodiment of the present invention, the output end of the electrically conductive terminal of the boost circuit board is inserted into the combining portion of the fiber bundle before or after the sleeve component is sheathed onto the fiber bundle.

According to an embodiment of the present invention, the electrically conductive adhesive is poured into the accommodating space before or after the output end of the electrically conductive terminal of the boost circuit board is inserted into the combining portion of the fiber bundle.

According to an embodiment of the present invention, the electrically conductive adhesive is poured into the accommodating space before or after the sleeve component is sheathed onto the fiber bundle.

According to an embodiment of the present invention, the sleeve component is a hollow pipe structure.

According to an embodiment of the present invention, the sleeve component is made of plastic material.

According to an embodiment of the present invention, the fiber bundle includes a plurality of fibers.

According to an embodiment of the present invention, each of the plurality of fibers is made of carbon material.

According to an embodiment of the present invention, the electrically conductive adhesive includes silver powders and thermosetting plastic.

In summary, in the present invention, the electrically conductive adhesive adhered between the combining portion of the fiber bundle and the output end of the electrically conductive terminal of the boost circuit board can not only adhere the combining portion of the fiber bundle to the output end of the electrically conductive terminal of the boost circuit board for improving a structural strength of a connection of the combining portion of the fiber bundle and the output end of the electrically conductive terminal of the boost circuit board but also be used as an electrically conductive medium between the combining portion of the fiber bundle and the output terminal of the electrically conductive terminal of the booster circuit board for solving a conventional problem of failure of emission of negative ions caused by a poor contact between the fiber bundle and the booster circuit board in the prior art.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 and FIG. 2 are partial diagrams of a negative ion generator at different views in the prior art.

FIG. 3 is a partial exploded diagram of the negative ion generator in the prior art.

FIG. 4 is a schematic diagram of a wearable air purifier according to an embodiment of the present invention.

FIG. 5 is an exploded diagram of the wearable air purifier according to the embodiment of the present invention.

FIG. 6 and FIG. 7 are enlarged diagrams of an A portion of the negative ion generator shown in FIG. 5 at different views according to the embodiment of the present invention.

FIG. 8 is a sectional diagram of the negative ion generator according to the embodiment of the present invention.

FIG. 9 and FIG. 10 are exploded diagrams of the negative ion generator at different views according to the embodiment of the present invention.

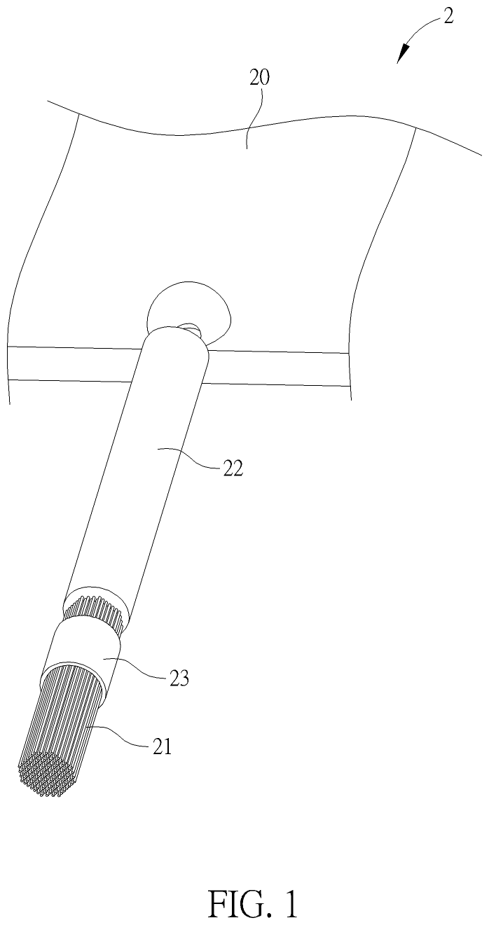

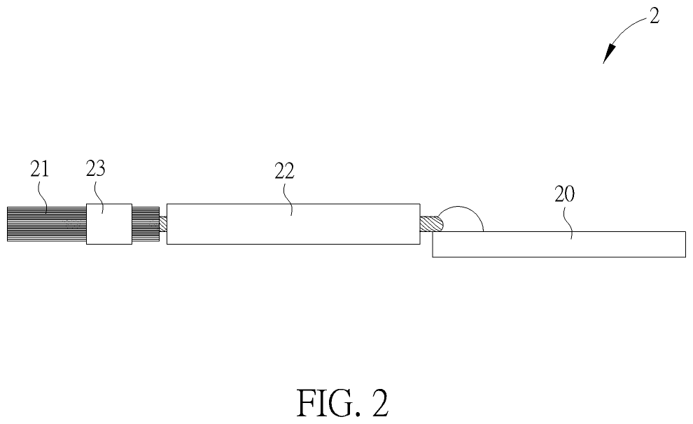

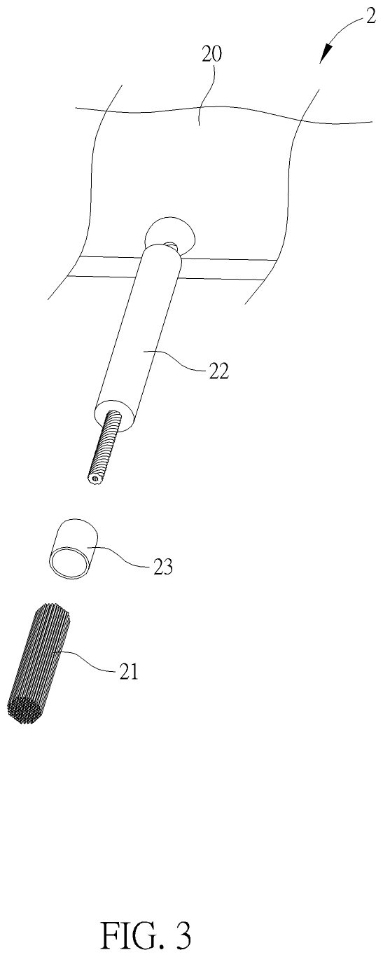

### DETAILED DESCRIPTION

In the following detailed description of the preferred embodiments, reference is made to the accompanying drawings which form a part hereof, and in which is shown by way of illustration specific embodiments in which the invention may be practiced. In this regard, directional terminology, such as “top,” “bottom,” “front,” “back,” etc., is used with reference to the orientation of the Figure (s) being described. The components of the present invention can be positioned in a number of different orientations. As such, the directional terminology is used for purposes of illustration and is in no way limiting. Accordingly, the drawings and descriptions will be regarded as illustrative in nature and not as restrictive.

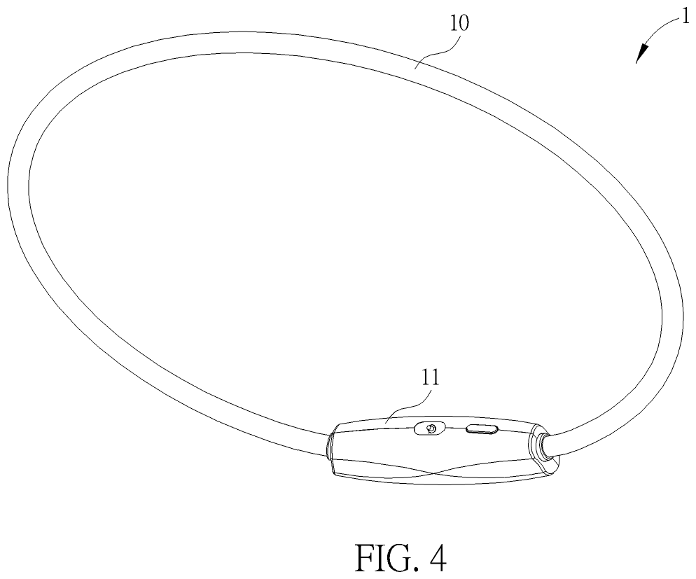

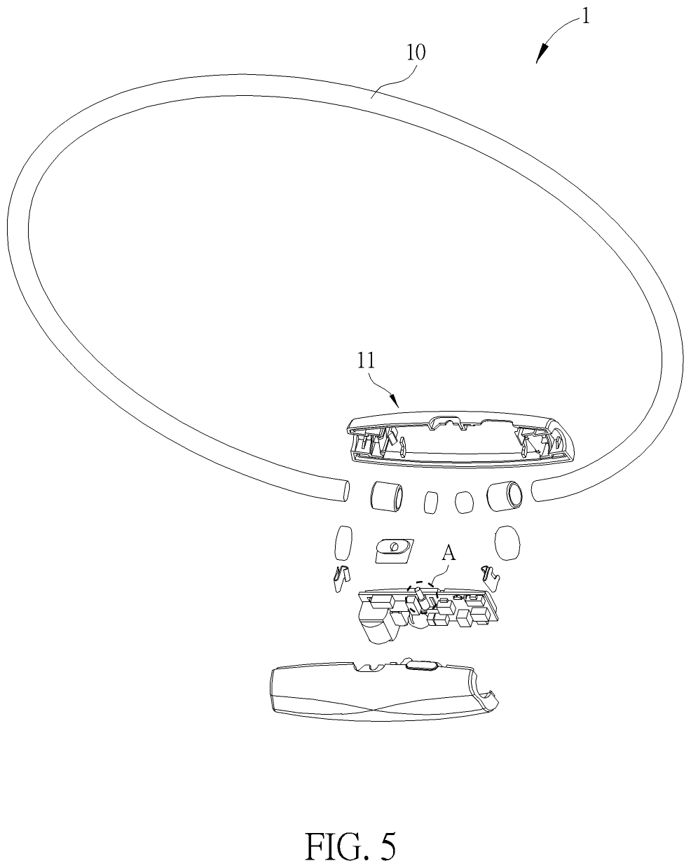

Please refer to FIG. 4 and FIG. 5. FIG. 4 is a schematic diagram of a wearable air purifier 1 according to an embodiment of the present invention. FIG. 5 is an exploded diagram of the wearable air purifier 1 according to the embodiment of the present invention. As shown in FIG. 4 and FIG. 5, the wearable air purifier 1 includes a wearable component 10 and a negative ion generator 11. The negative ion generator 11 is for emitting or generating negative ions. The wearable component 10 is detachably connected to the negative ion generator 11 for allowing a user to wear, which brings convenience in carrying the negative ion generator 11. However, the present invention is not limited thereto. In other words, the negative ion generator of the present invention also can be adapted to other electronic apparatuses. For example, in another embodiment, the negative ion generator can be combined with a hair dryer or a fan.

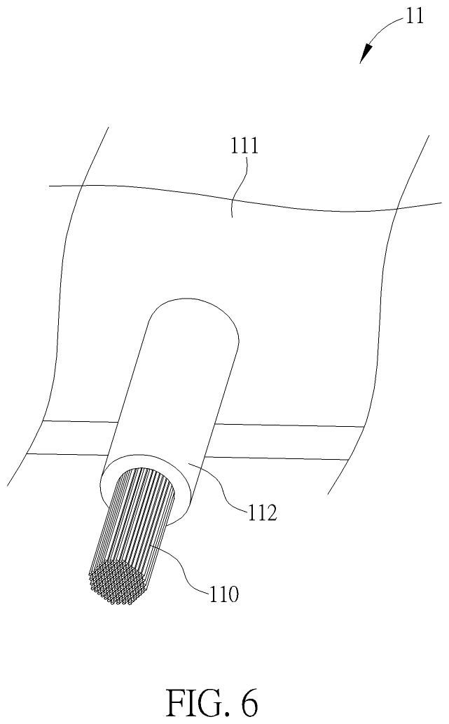

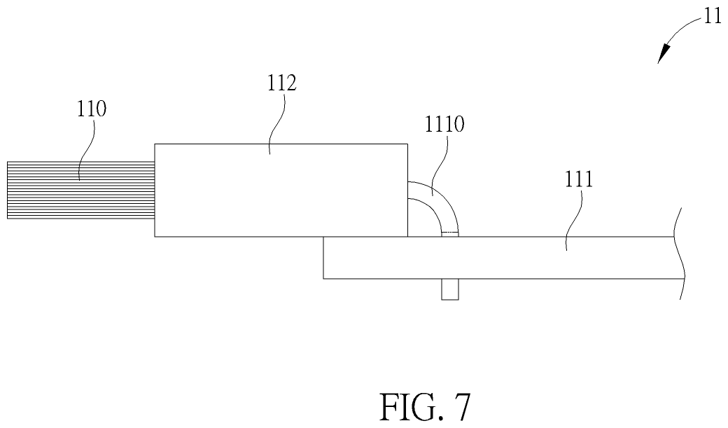

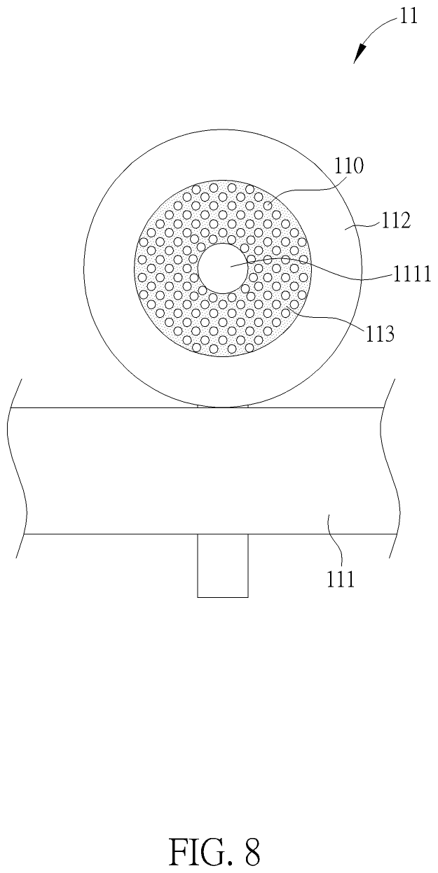

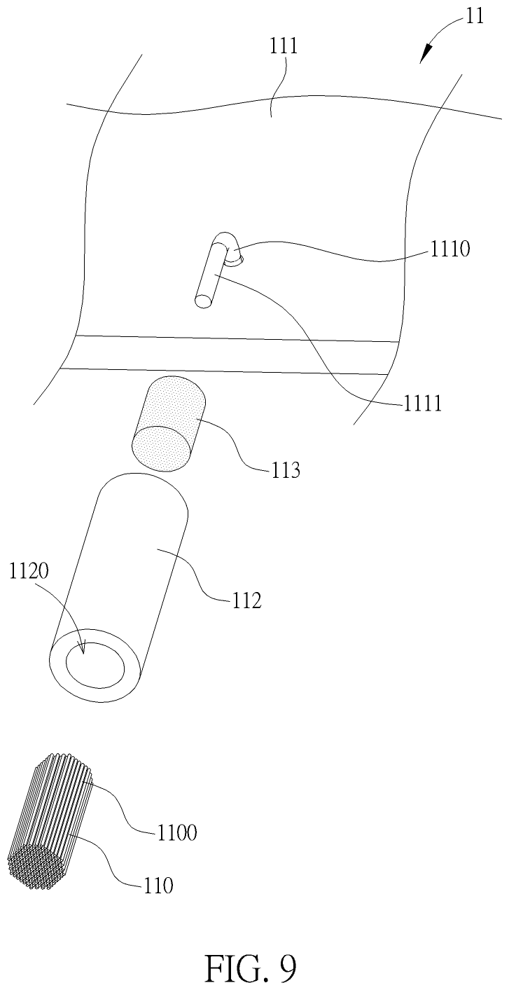

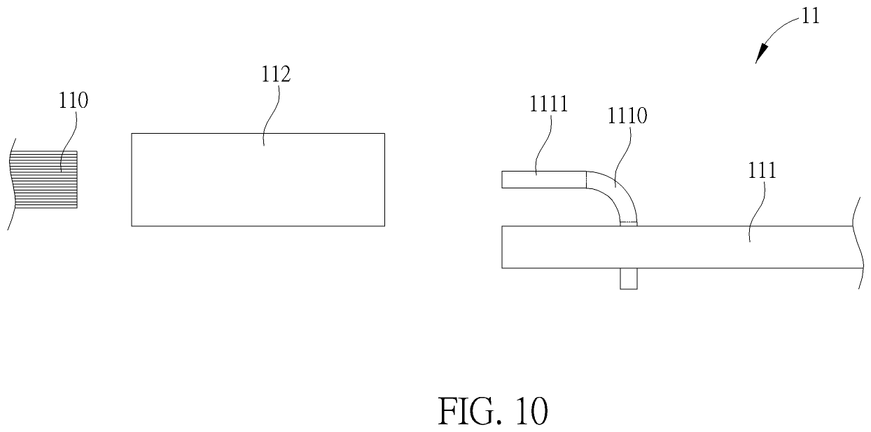

Please refer to FIG. 6 to FIG. 10. FIG. 6 and FIG. 7 are enlarged diagrams of an A portion of the negative ion generator 11 shown in FIG. 5 at different views according to the embodiment of the present invention. FIG. 8 is a sectional diagram of the negative ion generator 11 according to the embodiment of the present invention. FIG. 9 and FIG. 10 are exploded diagrams of the negative ion generator 11 at different views according to the embodiment of the present invention. In order to illustrate structure and operational principle of the present invention, FIG. 6 to FIG. 10 only show part of the negative ion generator 11. As shown in FIG. 6 to FIG. 10, the negative ion generator 11 includes a fiber bundle 110, a boost circuit board 111, a sleeve component 112 and an electrically conductive adhesive 113. The fiber bundle 110 includes a combining portion 1100. The boost circuit board 111 is connected to the fiber bundle 110. The boost circuit board 111 includes an electrically conductive terminal 1110. An output end 1111 of the electrically conductive terminal 1110 is inserted into the combining portion 1100 of the fiber bundle 110. The boost circuit board 111 is for providing a high-voltage current to the fiber bundle 110 to enable the fiber bundle 110 to emit negative ions by corona discharging. An accommodating space 1120 is enclosed by the sleeve component 112. The combining portion 1100 of the fiber bundle 110 is installed inside the accommodating space 1120. The electrically conductive adhesive 113 is poured into the accommodating space 1120 and located between the combining portion 1100 of the fiber bundle 110 and the output end 1111 of the electrically conductive terminal 1110 for adhering and electrically connecting the fiber bundle 110 to the electrically conductive terminal 1110 by the electrically conductive adhesive 113.

In this embodiment, the wearable component 10 can be preferably a necklace, a collar or a wristband. The sleeve component 112 can be preferably a hollow pipe structure and made of plastic material, e.g., silicone rubber. The fiber bundle 110 includes a plurality of fiber 1102, and the fiber bundle 110 can preferably be a carbon brush with the plurality of fiber 1102 made of carbon material. The electrically conductive adhesive 113 can preferably include silver powders and thermosetting plastic. However, the type of the wearable component, the material and shape of the sleeve component, the material and structure of the fiber bundle, and composition of the electrically conductive adhesive are not limited to this embodiment.

When it is desired to connect the fiber bundle 110 to the boost circuit board 111, the sleeve component 112 can be sheathed onto the fiber bundle 110, so as to locate the combining portion 1100 of the fiber bundle 110 at the accommodating space 1120 enclosed by the sleeve component 112. Afterwards, the output end 1111 of the electrically conductive terminal 110 of the boost circuit board 111 can be inserted into the combining portion 1100 of the fiber bundle 110. After the output end 1111 of the electrically conductive terminal 1110 of the boost circuit board 111 is inserted into the combining portion 1100 of the fiber bundle 110, the electrically conductive adhesive 113 can be poured into the accommodating space 1120. The electrically conductive adhesive 113 poured into the accommodating space 1120 can be filled between the combining portion 1100 of the fiber bundle 110 and the output end 1111 of the electrically conductive terminal 1110 to adhere the fiber bundle 110 to the electrically conductive terminal 1110 for electrically connecting the electrically conductive terminal 1110 to the fiber bundle 110 by the electrically conductive adhesive 113, which achieves purposes of structural connection and electrical connection of the fiber bundle 110 and the electrically conductive terminal 1110 of the boost circuit board 111.

However, the sequence of the steps of the method of connection of the fiber bundle 110 and the electrically conductive terminal 1110 of the boost circuit board 111 of manufacturing the negative ion generator 11 is not limited to the aforementioned one. For example, in another embodiment, the output end 1111 of the electrically conductive terminal 1110 of the boost circuit board 111 can be inserted into the combining portion 1100 of the fiber bundle 110, and then the sleeve component 112 can be sheathed onto the fiber bundle 110 so that the combining portion 1100 of the fiber bundle 110 is located at the accommodating space 1120 enclosed by the sleeve component 112. Afterwards, the electrically conductive adhesive 113 can be poured into the accommodating space 1120 after the sleeve component 112 is sheathed onto the fiber bundle 110.

In another embodiment, the output end 1111 of the electrically conductive terminal 1110 can be inserted into the accommodating space 1120 enclosed by the sleeve component 112, and then the electrically conductive adhesive 113 can be poured into the accommodating space 1120. Afterwards, the combining portion 1100 of the fiber bundle 110 can be inserted into the accommodating space 1120 enclosed by the sleeve component 112 after the electrically conductive adhesive 113 is poured into the accommodating space 1120, so as to achieve insertion of the output end 1111 of the electrically conductive terminal 1110 of the boost circuit board 111 into the combining portion 1100 of the fiber bundle 110 and sheathing of the sleeve component 112 onto the fiber bundle 110.

In another embodiment, the sleeve component 112 can be sheathed onto the fiber bundle 110, so as to locate the combining portion 1100 of the fiber bundle 110 at the accommodating space 1120 enclosed by the sleeve component 112, and then the electrically conductive adhesive 113 can be poured into the accommodating space 1120. Afterwards, the output end 1111 of the electrically conductive terminal 1110 of the boost circuit board 111 can be inserted into the combining portion 1100 of the fiber bundle 110 after the electrically conductive adhesive 113 is poured into the accommodating space 1120.

In another embodiment, the electrically conductive adhesive 113 can be poured into the accommodating space 1120 enclosed by the sleeve component 112, and then the combining portion 1100 of the fiber bundle 110 and the output end 1111 of the electrically conductive terminal 1110 of the boost circuit board 111 can be inserted into the accommodating space 1120, which is filled with the electrically conductive adhesive 113, via two openings at two ends of the sleeve component 112 to achieve insertion of the output end 1111 of the electrically conductive terminal 1110 of the boost circuit board 111 into the combining portion 1100 of the fiber bundle 110.

In another embodiment, the electrically conductive adhesive 113 can be poured into the accommodating space 1120, and then the combining portion 1100 of the fiber bundle 110 and the output end 1111 of the electrically conductive terminal 1110 of the boost circuit board 111 can be inserted into the accommodating space 1120 via two openings at two ends of the sleeve component 112 to achieve insertion of the output end 1111 of the electrically conductive terminal 1110 of the boost circuit board 111 into the combining portion 1100 of the fiber bundle 110.

From the above, the sequence of the step of installing the fiber bundle 110 and the electrically conductive terminal 1110 inside the accommodating space 1120 enclosed by the sleeve component 112 and the step of pouring the electrically conductive adhesive 113 into the accommodating space 1120 to adhere the fiber bundle 110 to the electrically conductive terminal 1110 can depend on practical demands.

In contrast to the prior art, in the present invention, the electrically conductive adhesive adhered between the combining portion of the fiber bundle and the output end of the electrically conductive terminal of the boost circuit board can not only adhere the combining portion of the fiber bundle to the output end of the electrically conductive terminal of the boost circuit board for improving a structural strength of a connection of the combining portion of the fiber bundle and the output end of the electrically conductive terminal of the boost circuit board but also be used as an electrically conductive medium between the combining portion of the fiber bundle and the output terminal of the electrically conductive terminal of the booster circuit board for solving a conventional problem of failure of emission of negative ions caused by a poor contact between the fiber bundle and the booster circuit board in the prior art.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A negative ion generator comprising:
a fiber bundle comprising a combining portion;
a boost circuit board connected to the fiber bundle, the boost circuit board comprising an electrically conductive terminal, an output end of the electrically conductive terminal being inserted into the combining portion of the fiber bundle, the boost circuit board being for providing a high-voltage current to the fiber bundle to enable the fiber bundle to emit negative ions by corona discharging;
a sleeve component, an accommodating space being enclosed by the sleeve component, the combining portion of the fiber bundle being installed inside the accommodating space; and
an electrically conductive adhesive poured into the accommodating space and located between the combining portion of the fiber bundle and the output end of the electrically conductive terminal for adhering and electrically connecting the fiber bundle to the electrically conductive terminal by the electrically conductive adhesive.

2. The negative ion generator of claim 1, wherein the sleeve component is a hollow pipe structure.

3. The negative ion generator of claim 1, wherein the sleeve component is made of plastic material.

4. The negative ion generator of claim 1, wherein the fiber bundle comprises a plurality of fibers.

5. The negative ion generator of claim 4, wherein each of the plurality of fibers is made of carbon material.

6. The negative ion generator of claim 1, wherein the electrically conductive adhesive comprises silver powders and thermosetting plastic.

7. A wearable air purifier comprising:
a wearable component; and
a negative ion generator detachably connected to the wearable component, the negative ion generator comprising:
a fiber bundle comprising a combining portion;
a boost circuit board connected to the fiber bundle, the boost circuit board comprising an electrically conductive terminal, an output end of the electrically conductive terminal being inserted into the combining portion of the fiber bundle, the boost circuit board being for providing a high-voltage current to the fiber bundle to enable the fiber bundle to emit negative ions by corona discharging;
a sleeve component, an accommodating space being enclosed by the sleeve component, the combining portion of the fiber bundle being installed inside the accommodating space; and
an electrically conductive adhesive poured into the accommodating space and located between the combining portion of the fiber bundle and the output end of the electrically conductive terminal for adhering and electrically connecting the fiber bundle to the electrically conductive terminal by the electrically conductive adhesive.

8. The wearable air purifier of claim 7, wherein the wearable component is a necklace, a collar or a wristband.

9. A method of manufacturing a negative ion generator, the method comprising:
sheathing a sleeve component onto a fiber bundle, so as to locate a combining portion of the fiber bundle at an accommodating space enclosed by the sleeve component;
inserting an output end of an electrically conductive terminal of a boost circuit board into the combining portion of the fiber bundle; and
pouring an electrically conductive adhesive into the accommodating space, so as to adhere the fiber bundle to the electrically conductive terminal for electrically connecting the electrically conductive terminal to the fiber bundle by the electrically conductive adhesive.

10. The method of claim 9, wherein the output end of the electrically conductive terminal of the boost circuit board is inserted into the combining portion of the fiber bundle before or after the sleeve component is sheathed onto the fiber bundle.

11. The method of claim 9, wherein the electrically conductive adhesive is poured into the accommodating space before or after the output end of the electrically conductive terminal of the boost circuit board is inserted into the combining portion of the fiber bundle.

12. The method of claim 9, wherein the electrically conductive adhesive is poured into the accommodating space before or after the sleeve component is sheathed onto the fiber bundle.

13. The method of claim 9, wherein the sleeve component is a hollow pipe structure.

14. The method of claim 9, wherein the sleeve component is made of plastic material.

15. The method of claim 9, wherein the fiber bundle comprises a plurality of fibers.

16. The method of claim 15, wherein each of the plurality of fibers is made of carbon material.

17. The method of claim 9, wherein the electrically conductive adhesive comprises silver powders and thermosetting plastic.
